(19) United States
(12) Patent Application Publication
(10) Pub. No.: US 2026/0272615 A1
(43) Pub. Date: Sep. 17, 2026

(54) DRILL BIT AND METHOD TO PERFORM AN OSTEOTOMY USING THIS DRILL BIT

(71) Applicant: Nobel Biocare Services AG, Kloten (CH)

(72) Inventors: Jörg Weitzel, Rielasingen-Worblingen (DE); Curdin Schlumpf, Winterthur (CH)

(21) Appl. No.: 19/167,561

(22) PCT Filed: Mar. 13, 2024
(86) PCT No.: PCT/EP2024/056725

§ 371 Date: Sep. 22, 2025

(30) Foreign Application Priority Data
Mar. 23, 2023 (EP) 23163836.2

(51) Int. Cl. A61C 8/00; A61B 17/16

(52) U.S. Cl. CPC A61C 8/0089; A61B 17/1615

(57) ABSTRACT

The present disclosure provides a drill bit comprising an apical end, a coronal end, and a longitudinal axis extending between the apical end and the coronal end. The drill bit further includes a drill bit core extending along the longitudinal axis, at least two flutes, each flute being defined by a concave surface forming a recess along the drill bit core, and an apical end face at the apical end. Apical edges of the concave surfaces and an apical edge of the drill bit core define the shape of the apical end face so that the apical end face defines a profile of retention arms at the apical end, each retention arm being formed between the concave surfaces of adjacent flutes. The present disclosure also provides a method to create an osteotomy using a drill bit.

### CROSS REFERENCE TO RELATED APPLICATION

This application is a 371 National Stage of International Patent Application No. PCT/EP2024/056725, filed on Mar. 13, 2024, which claims priority to European Patent Application No. EP 23163836.2, filed Mar. 23, 2023, the disclosures of which are incorporated by reference herein in their entireties.

### TECHNICAL FIELD

The present disclosure relates to a drill bit for preparing an implantation site of a dental implant. It also relates to performing an osteotomy for preparing an implantation site.

### BACKGROUND

Before insertion of a dental implant, a recess in the upper or lower jawbone of a patient needs to be prepared. This osteotomy is an important step in providing a dental implant and has a significant influence on the osseointegration of the implant and its long-term success.

Given that the density, orientation, and quality of bone differs from patient to patient, it is often necessary to use various tools to prepare an appropriate implant-receiving recess. However, the more interdependent tools for creating a recess are employed, the more extensive the treatment protocol becomes. This renders the decision on the details of such a protocol rather complex and time-consuming. Moreover, the alignment of these various tools for preparing an implantation site may turn out to be rather difficult.

It has to be taken into account that the jawbone has a comparatively hard outer layer (i.e., the cortical bone) and underneath a weaker sponge-like bone structure (i.e., the cancellous bone). The cortical bone providing the hard cortex is much denser and less elastic than cancellous bone. When preparing a bone recess for receiving a dental implant, a drill bit has to generate this recess extending from the cortical bone into the cancellous bone. Thus, a drill bit should be capable of machining different types of bone tissue in a patient during an osteotomy.

During the creation of the osteotomy, the drill bit is guided by a dental professional. Accordingly, there is also an influence of the dental professional on performing the osteotomy.

In view of the above, there has been a constant effort to improve the process of creating a recess within bone tissue in preparation for the insertion of a dental implant. In this respect, WO 2017/129828 A1 discloses a drill bit for preparing a bone recess that is configured to concurrently condense and cut bone tissue.

### SUMMARY

It has generally been an objective to provide a drill bit that facilitates performing an osteotomy independent of varying bone quality in a jawbone of a patient as well as in different jawbones of a patient or between patients so that a bone recess for receiving the dental implant turns out as desired, i.e., the bone recess should neither be underprepared nor be overprepared.

It has also been an objective to provide feedback to the dental professional about the progress of preparing the implantation recess.

With these objectives in mind, a drill bit and a method to perform an osteotomy are provided as defined in the independent claims. The dependent claims specify preferred features of the claimed invention.

The present disclosure according to a first aspect provides a drill bit comprising an apical end, a coronal end, and a longitudinal axis extending between the apical end and the coronal end. Further, the drill bit includes a drill bit core extending along the longitudinal axis, at least two flutes, each flute being defined by a concave surface forming a recess along the drill bit core, and an apical end face at the apical end. Apical edges of the concave surfaces and an apical edge of the drill bit core define the shape of the apical end face so that the apical end face defines a profile of retention arms at the apical end, wherein each retention arm is formed between the concave surfaces of adjacent flutes. At least one of the at least two flutes extends, along the longitudinal axis, from the apical end, on at least 70% of the length of the drill bit configured to interact with bone tissue, preferably over the full length of the drill bit configured to interact with bone tissue.

Along the longitudinal axis, the linear distance, between the most apical point of the at least one flute and the most coronal point of said flute is at least 50% of the total linear distance of the drill bit.

Preferably, the at least one flute extends, along the longitudinal axis L, over the full length of the (operative part of) drill bit. In other words, a first extremity of at least one flute is at an apical end of the drill bit and a second extremity of said flute is at the coronal end of the drill bit. The at least one flute can extend continuously between said first and second extremities. The drill bit can comprise two or more flutes having the same above-mentioned features.

Having at least one cutting flute extending on a significant portion of the drill bit allows the cutting of bone tissue along the entirety of the flute. The shape/volume of the osteotomy created by the cutting flute may substantially correspond to the shape/volume of the implant core. When an implant is subsequently placed in the prepared osteotomy, only the thread (or threads) of the implant (and not the core of the implant) compresses the bone tissues. This results in preservation of a maximum of bone tissues. In other words, when the implant is placed in the osteotomy only the implant thread pushes the bone radially relative to the implant axis. A large portion of the bone tissues is therefore preserved from destructive over-compression and ultimately this leads to better osseointegration. This is particularly important for dense bone-tissues as, over-compression damages dense bone tissue and can even fracture a portion of a patient jaw. The drill bit according to the invention is particularly adapted to prepare an osteotomy in this type of hard bone.

The above-mentioned advantages are increased in an embodiment of the present invention wherein at least one cutting flute extend along the full length of the drill bit.

Each flute extends from the apical end face towards the coronal end. In a cross-section perpendicular to the longitudinal axis, the concave surface of a flute forms a concavity along the profile of the drill bit core. At the apical end face, each flute causes a concavity in the profile of the end face.

Between adjacent concavities, the profile of the end face is arm-shaped and represents a profile of a retention arm. This profile preferably has a longitudinal extension in a radial direction (relative to the longitudinal axis) and extends from a central area of the apical end face to the outer edge of the apical end face (generally defined by an apical edge of the drill bit core). The number of retention arms equals the number of flutes. Preferably, the drill bit comprises three flutes.

The end face provides a bluntness to the tip of the drill bit resulting at least in a reduced forward cutting capability of the drill bit. In other words, the end face increases the force and torque needed to advance the drill bit further upon contact of the apical end face with bone tissue.

Further, the drill bit core is preferably tapered in the apical direction of the drill bit along at least a portion of the drill bit core. This tapered portion serves to increase the diameter of the hole in the bone tissue upon advancement of the drill bit. Additionally, the cutting flute extending along this tapered portion of the drill bit cuts bone tissues at each point of its cutting edge and creates an osteotomy or hole. The osteotomy is enlarged at each point by the shape of the drill and prepared to the size of the dental implant.

Upon contact of the apical end face with the bottom of a pilot hole in the bone tissue of the implantation site, the retention arms are configured to interact with the bone tissue creating a retention force that counteracts a driving torque applied to the drill bit. In other words, the retention arms cause a frictional force with bone tissue at the bottom of the pilot hole upon contact, in particular surface contact, that resists the driving torque applied to the drill bit.

As a result, the retention arms at the apical end of the drill bit provide feedback indicating to a user that the end of a previously prepared hole (i.e., pilot hole) within the bone tissue has been reached and, thus, serve to prevent the osteotomy to be underprepared or overprepared.

The drill bit may further comprise at least one guiding thread protruding from the drill bit core. The guiding thread extends helically along the drill bit core and preferably tapers apically. The guiding thread is in particular a single thread.

The guiding thread of the drill bit allows for an enhanced control of the drill bit advancing into the bone tissue since the guiding thread converts a rotation of the drill bit into a predetermined feed rate. More specifically, each rotation advances the drill bit by a distance that corresponds to the pitch of the guiding thread. In this way, the guiding thread particularly facilitates insertion of the drill bit into a previously prepared pilot hole at the implantation site.

The guiding thread may taper in the apical direction of the drill bit. That is, the outer diameter of the guiding thread decreases in a coronal-apical direction. Preferably, the thread height of the guiding thread is substantially constant in the direction of the longitudinal axis. It may also or alternatively decrease in height in this direction. Preferably, the guiding thread does not extend to the apical end of the drill bit or to the portion, where the retention arms are arranged.

The guiding thread is preferably formed so that the forward driving force resulting from converting torque into an advancement of the drill bit into a previously prepared hole is insufficient to advance the drill bit into unprepared bone tissue. Instead, the guiding thread loses grip when reaching a certain torque limit and starts rotating freely without drilling deeper into the tissue.

Accordingly, the guiding thread may enhance above-noted effect of providing feedback when reaching the end of a previously drilled hole or pilot hole by increasing the torque until the thread loses grip and is spinning. Thus, the apical end face, the retention arms, and the guiding thread interact with each other in providing a counteracting feedback torque.

The radial height of the guiding thread is particularly smaller than the radial extension of the retention arm's profile at the apical end face.

As described above, the profile of the retention arms at the apical end face extends from a central area to the outer edge of the apical end face. The length of the profile from the central area to the outer edge is preferably greater than the radial height of the guiding thread. In general, longer retention arms increase the counteracting torque when the drill bit reaches the bottom of a previously drilled hole.

Each retention arm may have a radial extension that is greater than the circumferential width at its tip formed by the apical edge of the drill bit core.

The guiding thread may have a non-circular outer circumferential profile, the non-circular outer circumferential profile preferably being a trioval profile.

A non-circular and, in particular, a trioval profile has the advantage to act as a guiding thread that forms the thread grooves more easily than a regular thread. It also acts by gradually condensing the bone tissue at the thread path of the guiding thread. In this way, bone tissue may be preserved instead of being cut which reduces the impact of the guiding thread on the shape of the hole formed by the drill bit.

Further, the drill bit core may have a non-circular profile in a cross-section perpendicular to the longitudinal axis, wherein in the longitudinal direction of the drill bit, the non-circular profile preferably rotates around the longitudinal axis creating a twisted non-circular drill bit core.

Such a non-circular profile of the drill bit core uses the elasticity of the bone tissue when creating a recess in the bone tissue upon insertion of the drill bit while the drill bit is rotating. Without the wish of being bound by theory, the non-circular profile assists in creating a recess that allows the drill bit to keep its direction of insertion. It may also have a positive effect on primary stability of a dental implant that is inserted into this recess due to a condensing effect on the bone tissue. The non-circular profile of the drill bit core may also provide the core with a relief angle helping in the cutting of the bone tissue.

A twisted non-circular drill bit core has the advantage to distribute the forces resulting from the non-circular profile upon insertion of the drill bit into bone tissue along its circumference. This particularly prevents the drill bit from being offset when entering the pilot hole.

The non-circular profile of the drill bit core and the non-circular profile of the guiding thread may be formed with a circumferential offset. Such a configuration also enhances the distribution of forces along the circumference of the drill bit and prevents the drill bit to become misaligned or offset in relation to its desired trajectory of insertion.

Preferably, an apical edge or circumferential edge of the apical end face is arranged in a plane perpendicular to the longitudinal axis.

This structural feature results in the retention arms contacting the bottom of a previously drilled hole at essentially the same time. As a result, the counteracting torque increase more rapidly and provides clearer feedback. In this respect, at least the apical surface of the retention arms and preferably the apical end face is flat to cause a surface contact with the bone tissue at the bottom of the pilot hole.

The concave surface of each flute and the circumferential surface of the drill bit core preferably form a cutting edge. If a guiding thread is provided, the cutting edge even more preferably also includes the profile of the guiding thread.

Accordingly, such a flute does not only form the retention arms at the apical end face but may also be a cutting flute that assists in preparing a recess in bone tissue that substantially corresponds to the rotational shape of the drill bit core. The cutting edge facilitates forming the bone recess and further enables the retention arms and, if present, the guiding thread, to cause a more detectible increase in torque that resists further rotation of the drill bit upon contact with the bottom of the previously drilled hole.

The concave surface and the apical end face may at least partially form a cutting edge at the apical edge of each concave surface, the cutting edge having a forward rake angle and substantially no forward relief angle.

Since there is basically no forward relief angle at the edge formed at the apical end face of the drill bit, the forward cutting capability of the apical end face is significantly reduced.

Further, the forward rake angle is relatively large due to the flutes extending in the longitudinal direction of the drill bit. As a result, the surface of the retention arms at the edges formed between the concave surfaces and the apical end face, the surface facing in a predetermined rotational direction of the drill bit, collects or scrapes bone tissue at the bottom of previously drilled hole. This bone tissue tends to create friction and a torque that counteracts the rotation of the drill bit and, thus, provides feedback.

The at least two flutes particularly extend helically along the drill bit core. The angle between the apical end face of the drill bit and the concave surface of the flute, the angle being formed due to the helical extension of the flute, supports the functionality of the retention arms by collecting or scraping off bone tissue at the apical edge, where the apical end face and the concave surface are joined. Further, the helical path of the flutes provides a more uniform load distribution along the circumference of the drill bit. This is particularly advantageous when drilling with a slow speed and, thus, results in a controlled preparation of the bone recess or implantation recess. In this respect, three flutes are preferred.

Further, the present disclosure provides a method to create an osteotomy using a drill bit, in particular a drill bit as described above. The method comprises the steps of drilling a hole with a pilot drill and increasing the size of the hole for the insertion of a dental implant using the drill bit. The drill bit comprises a drill bit core, at least two flutes, wherein each flute is defined by a concave surface forming a longitudinal recess along the drill bit core, an apical end face at the apical end of the drill bit, and retention arms extending coronally from the apical end face. Each retention arm is formed between concave surfaces of adjacent flutes. Upon contact of the apical end face with the bottom of the hole drilled with the pilot drill, the retention arms generate a braking force that may be used for stopping the rotation of the drill bit.

Upon contact with the bottom of the hole, the apical end face acts as a stop. Further, the rotating retention arms collect bone tissue or bone debris at the bottom causing a torque that counteracts the driving torque of the drill bit. This provides feedback, either to a dental professional using the drill bit or to a sensor or mechanism such as a torque limiter as a signal to stop the drilling process.

Preferably, the tip of the pilot drill has a diameter greater than the diameter of the apical end face. This has the advantage that at the beginning of preparing the bone recess using the drill bit, the apical end face does not get into contact with bone tissue until the drill bit reaches the end of the pilot hole. Otherwise, the apical end face of the drill bit would hinder the drilling process due to its forward drilling capability being at least significantly reduced. Further, the torque signal indicates the end of preparation more clearly.

The diameter at the tip of the pilot drill refers to the diameter at the border between frontal cutting edges formed due to the point angle of the pilot drill (if present) and the circumferential surface extending in a direction along the longitudinal axis of the pilot drill.

### SHORT DESCRIPTION OF THE DRAWINGS

The following figures illustrate preferred embodiments of features in accordance with the present disclosure. These embodiments are not to be construed as limiting but are merely for enhancing the understanding of the disclosure.

FIG. 1 is a partial side view of a drill bit according to the present disclosure;

FIG. 2 is a partial view of the drill bit depicted in FIG. 1 illustrating the apical end of the drill bit in perspective;

FIG. 3 is a frontal view of the apical end face of the drill bit;

FIG. 4 is a cross-sectional view of a drill bit according to the present disclosure;

FIG. 5 is another cross-sectional view of a drill bit according to the present disclosure; and

FIG. 6 is yet another cross-sectional view of a drill bit according to the present disclosure.

### DETAILED DESCRIPTION OF PREFERRED EMBODIMENTS

In the following, exemplary embodiments of a tool for preparing a recess in bone tissue and, in particular, embodiments of a drill bit 1 according to the present disclosure will be described under reference to the accompanying figures.

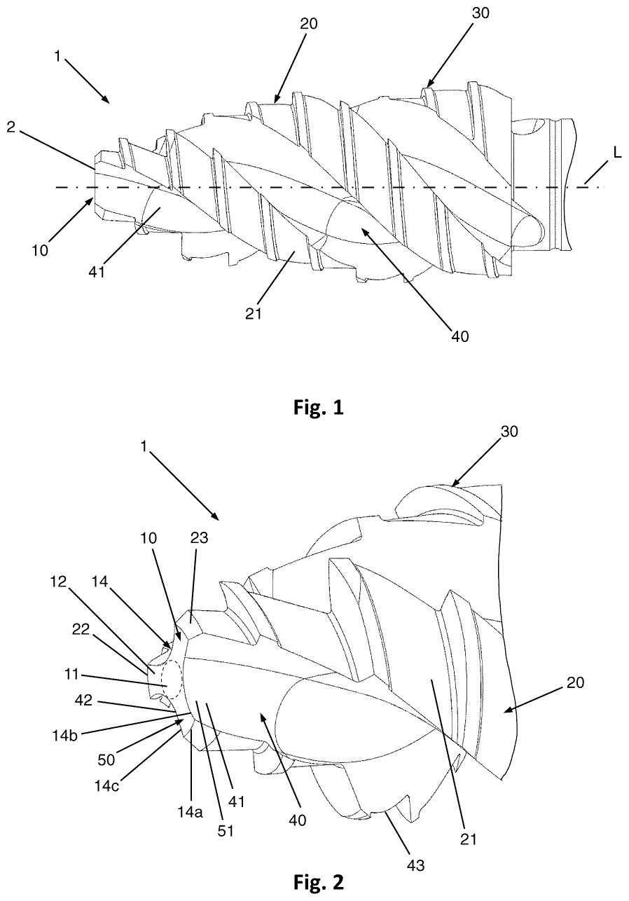

FIG. 1 shows an exemplary embodiment of a drill bit 1 according to the present disclosure. The drill bit 1 has an apical end 2 and a coronal end (not shown). It extends along a longitudinal axis L that also acts as a rotational axis. The coronal end is preferably configured to be coupled to a dental drill (not shown) for transmitting a driving torque to the drill bit 1.

The drill bit 1 includes a drill bit core 20 with a circumferential surface 21. The drill bit core 20 preferably tapers in a coronal-apical direction (i.e., towards the apical end 2). The drill bit 1 comprises at least two flutes 40. Each flute 40 is defined by a concave surface 41. The concave surface 41 forms a longitudinal recess (i.e., a flute 40) that starts at the apical end 2 and extends along the drill bit core 20. Accordingly, each flute 40 has a concave profile defined by the concave shape of the concave surface 41.

The drill bit core 20 preferably has a substantially continuous profile along the longitudinal axis L of the drill bit 1. The profile of the drill bit core 20 is rendered “substantially” continuous due to the flutes 40 and a guiding thread 30 (if present). In other words, the profile does not comprise a discontinuity, for example a discontinuity that is basically arranged perpendicular to the longitudinal axis L such as a step in the diameter of the drill bit core 20.

At the apical end 2, the drill bit 1 includes an apical end face 10. The apical end face 10 is preferably oriented substantially perpendicular to the longitudinal axis L of the drill bit 1. As illustrated in FIG. 2, the profile of the apical end face 10 is defined by the apical edges 42 of the concave surfaces 41 and the apical edge 22. At the apical edge 22 of the drill bit core 20, the drill bit core 22 may include a chamfer or rounded edge 23. The chamfer 23 does not form part of the apical end face 10.

The apical edges 42 of the concave surfaces 41 and the apical edge 22 of the drill bit core 20 provide a shape to the profile of the apical end face 10 that includes arm-like radial extensions 12 (in the exemplary embodiment of FIG. 2 the apical end face 10 has three arms). Each of these arm-shaped radial extensions 12 has a tip edge 14a defined by the apical edge 22 of the drill bit core 20 and two side edges 14b and 14c that are defined by apical edges 42 of concave surfaces 41 of two adjacent flutes 40. In particular, the opposite side edges 14b and 14c of each arm 12 may be defined by substantially half of the length of an apical edge 42 of a flute 40. Preferably, each arm-shaped extension 12 is substantially symmetrical relative to a radial axis of symmetry extending from the longitudinal axis L.

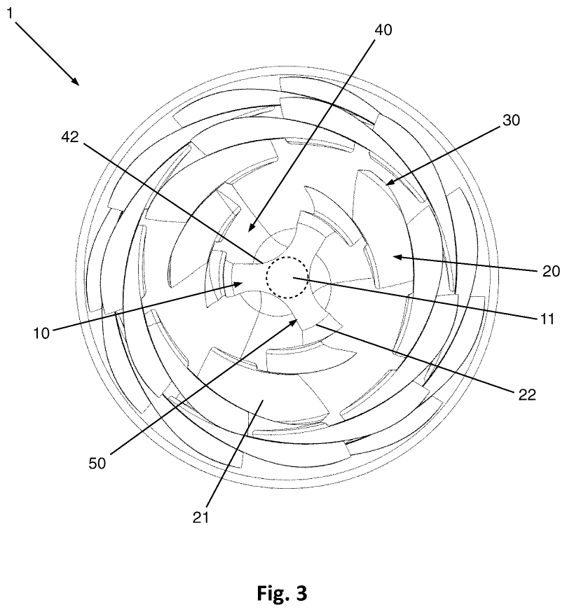

The arm-shaped extensions 12 extend from a central area 11 of the apical end face 10. Preferably, the central area 11 is defined by a virtual circle (in FIGS. 2 and 3 shown as a circle with dashed lines) that is fitted to the apical edges 42 of the concave surfaces 41.

Each of the arm-shaped extensions 12 defines a profile of a retention arm 50 for collecting bone debris or for scraping off bone tissue upon contact with bone tissue at the bottom of a pilot hole. Such a pilot hole is created prior using the drill bit 1 for preparing a bone recess that may be fitted for inserting a dental component such as a dental implant.

Each retention arm 50 extends from the apical end face 10 along a portion of the drill bit core 20 at the apical end 2 of the drill bit 1. Upon rotation of the drill bit 1, a side 51 of each retention arm 50 that faces in the direction of rotation collects bone debris which, in turn, counteracts the driving torque applied to the drill bit 1 (e.g., by a dental handpiece). Accordingly, the apical portion of the drill bit core 20 comprising the retention arms extends along a distance of the drill bit suitable for collecting bone tissue at the bottom of a pilot hole. Each retention arm 50 extends longitudinally from the apical end face 10 until the corresponding guiding thread 30.

In general, the longer the radial extension of the retention arms 15, the higher the torque created by scraping off or collecting bone tissue that counteracts the driving torque driving the drill bit 1.

If a pilot hole is formed as a blind hole (i.e., with a flat bottom), the retention arms 50 are collecting and accumulating bone debris when the apical end face 10 is in contact with the bottom of the pilot hole and the drill bit 1 is rotated.

If the drill used for drilling a pilot hole has a conical tip, the pilot hole tapers at its end. Due to this taper, the drill bit 1 will collect and accumulate bone debris upon contact with the conical wall of the bottom of the pilot hole. The drill bit 1 will also advance further into the pilot hole reshaping its bottom to be a blind hole. This results in the apical end face 10 getting into surface contact with bone tissue.

Thus, independent of the shape of the bottom of the pilot hole, the drill bit 1 will show an increase in torque upon reaching the end of the pilot hole. This increase in torque may serve as a feedback signal to stop the drilling process. For example, this increase in torque may activate a torque limiting device that automatically stops rotation of the drill bit 1.

Accordingly, such a design of the drill bit 1 may ensure that the recess created by the drill bit 1 is neither underprepared nor overprepared.

Further, upon surface contact of the apical end face 10 of the drill bit with bone tissue, the apical end face 10 is essentially designed without a forward drilling capability, i.e., the apical face 10 of the drill bit 1 has essentially no significant capability to cut into a surface of bone tissue that is arranged parallel to the apical end face 10.

In this respect, a flat surface of the arms basically causes the apical end face 10 to have no forward relief angle (i.e., a relief angle of 0°) at the apical edge 42 of each flute 40. Depending on the trajectory of extension of the flutes 40 along the drill bit core 20, the apical end 2 of the drill bit 1 may also lack a forward rake angle at the apical edge 42 of each of the flutes 40 (i.e., it has a rake angle of 0°).

Preferably, the flutes 40 extend helically about and along the longitudinal axis L along the drill bit core 20. Here, the apical edges 42 of the flutes 40 may have a non-zero forward rake angle. However, this forward rake angle is rather small (preferably less than 20°, 15°, 10°, or 5°). As a result, the apical edges 42 tend to mostly scrape off and collect bone tissue and, thus, contribute to creating a torque that counteracts the driving torque of the drill bit 1.

The flutes 40 may also change their pitch in an apical-coronal direction. Particularly, their pitch may decrease in this direction so that the forward rake angle at the apical edges 42 is reduced.

The exemplary embodiment illustrated in the figures comprises three flutes. Alternatively, two, four, or five flutes 40 may be provided. At the apical end 2 of the drill bit 1, these flutes 40 define two, four, or five arms, respectively. It has been found that a number of three flutes 40 is particularly advantageous for preparing the bone recess with the drill bit 1.

The flutes 40 preferably form a cutting edge 43 with the drill bit core 14. More specifically, the surface side of the flute facing in the direction of rotation forms a cutting edge with the circumferential surface 21 of the drill bit core 20. Accordingly, the drill bit 1 has a radial cutting capability upon its rotation.

As described above, at least a portion of the drill bit core 20 may be tapered in a coronal-apical direction. In combination with the flutes 40, this provides a forward cutting capability to the drill bit 1. In other words, such a configuration of a drill bit 1 cuts bone tissue when being advanced into bone tissue so that insertion of the drill bit 1 is facilitated.

Nonetheless, such a forward cutting capability of the drill bit 1 due to the interaction of the concave surfaces 41 and the circumferential surface 21 of the drill bit core 20 does not affect the limited cutting capability of the apical end face 10 at the apical end 2 due to its blunt configuration. Thus, the cutting capability of the cutting edges 43 at the edges of the circumferential surface 21 and the concave surfaces 41 serves for providing the bone recess with a desired shape.

As shown in the accompanying figures, the drill bit 1 may include a guiding thread 30. The guiding thread radially extends from the drill bit core 20 and extends at least along a portion of the drill bit core 20 about and along the longitudinal axis L. The guiding thread 30 preferably does not extend to or within the portion comprising the retention arms 50 at the apical end 2 of the drill bit.

The guiding thread 30 extends along the drill bit core 20 and is configured to pull the drill bit 1 into bone tissue with a predetermined rate. This rate depends on the ratio of rotation to advancement which is set by the pitch of the guiding thread 30. Preferably, the pitch of the guiding thread 30 is in a range of a 0.4 mm to 3 mm, more preferably in a range of 0.8 to 1.5 mm and even more preferably in a range of of 1.0 to 1.2 mm. The guiding thread 30 is preferably configured as a single thread but may also be designed as a double or triple thread. The guiding thread 30 provides a self-drilling capability to the drill bit 1.

The guiding thread 30 is preferably configured to create an advancement force that is smaller than an advancement force needed to overcome the resistance of the apical end face 10 upon surface contact of the apical end face 10 with the bone tissue of the bottom of the pilot hole.

In a cross-section, the guiding thread 30 has a thread profile with an average thread height from the thread ground to the tip of the guiding thread 30 that is smaller than the diameter of the apical end face 10 of the drill bit 1 (cf. FIGS. 2 and 3). The average thread height of the guiding thread is particularly in a range of 1% or 2%, to 5%, 10%, or 30% of the diameter of the apical end face 10.

Further, the maximum height of the guiding thread 30 may not exceed 50% of the diameter of the apical end face 10.

The height of the guiding thread 30 has the effect that upon contact of the apical end face 10 with the bottom of a pilot hole, the advancement force generated by the guiding thread 30 is insufficient to overcome the resistance against the advancement of the drill bit 10 caused by a surface contact of the apical end face 10 with the bottom of the pilot hole. As a result, an increase in torque occurs and may be used as a feedback signal for a user, a sensor, a mechanism, etc. to stop the application of a driving torque to the drill bit 1 or to indicate that the bottom of the pilot hole has been reached.

In other words, once apical end face 10 of the drill bit 1 gets in contact with bone tissue at the bottom of a pilot hole, the driving torque is required to increase for enabling a continuous rotation of the drill bit 1. In particular this required increase in torque may be detected and be used as a feedback signal.

For example, an increase in torque may automatically be generated by a motor, in particular an electric motor, driving the drill bit 1. Preferably a torque limiter is included in the drive train of the drill bit 1 that will react to such an increase by stopping the rotation of the drill bit 1. Otherwise, such an increase in driving torque may cause the guiding thread 30 to slip or spin. Even in such a scenario the advancement of the drill bit 1 may be stopped upon contact between the bottom of the pilot hole and the apical end face of the drill bit 1.

As previously described and illustrated in the figures, the guiding thread 30 may be formed at least partially as a tapered thread tapering in a coronal-apical direction (i.e., apically). The guiding thread 30 is preferably tapered along a portion, where the drill bit core 20 is also tapered. Further, the taper of the guiding thread 30 and the taper of the drill bit core 20 preferably correspond to each other. In other words, the taper of the guiding thread 30 and the taper of the drill bit core 20 preferably have the same angle in relation to the longitudinal axis L or run along parallel paths in a cross-section along the longitudinal axis L.

Further, the guiding thread 30 may have a non-circular profile, in particular a tri-oval profile. Such a non-circular profile of a guiding thread 13 may also be twisting around the longitudinal axis L along the drill bit 1.

A non-circular profile has the advantage, in particular in combination with the flutes 40, to create a thread path in bone tissue based on cutting and condensing. In other words, the tread path is partly created based on an elastic deformation of bone tissue. Due to the creation of a thread path based on elastic deformation of bone tissue, the apical end face 10 reaching the end or bottom of the pilot hole may cause an increase in torque over a longer time period since even upon spinning, the guiding thread 30 of the drill bit 1 may tend to deform bone tissue rather than cutting it away once the drill bit 1 starts spinning.

Additionally or alternatively, the drill bit core 20 may at least partly have a non-circular profile in cross sections perpendicular to the longitudinal axis L. Such a configuration of the drill bit core 20 may also be twisted, i.e., the non-circular profile rotates about the longitudinal axis L in cross sections along the coronal-apical direction. Further, and if present, a non-circular profile guiding thread 30 and a non-circular profile drill bit core 20 may be twisted in relation to each other (i.e., either or both are twisted about the longitudinal axis).

At the apical end (i.e., at the apical end face) the drill bit core 20 and preferably the portion along the drill bit core 20 forming the retention arms 50 may have a circular profile. This circular profile changes coronally to the non-circular profile.

The non-circular profile of the drill bit core may cause the drill bit 1 to remain at its position along its trajectory when preparing the bone recess upon the apical end face 10 reaching the bottom of the pilot hole. The non-circular profile in combination with the flutes 40 including the cutting edge 43 are configured to form a bone recess based on cutting bone tissue and elastic deformation of bone tissue. The elastic deformation may keep the drill bit 1 in position upon bone tissue contact of the apical end face 10. If the drill bit 1 keeps rotating, it may also prevent wiggling of the drill bit 1 and, thus, further cutting action of the drill bit 1.

The effects of a non-circular profile on forming a recess and on the cutting behavior of a drill bit are described in more detail in EP 21 208 105 A, which is herewith incorporated by reference.

More specifically, the relationship between the flutes 40 and the non-circular cross-section of the drill bit core 20 advantageously affects the way, the drill bit 1 forms the desired bone recess by cutting and deforming bone tissue.

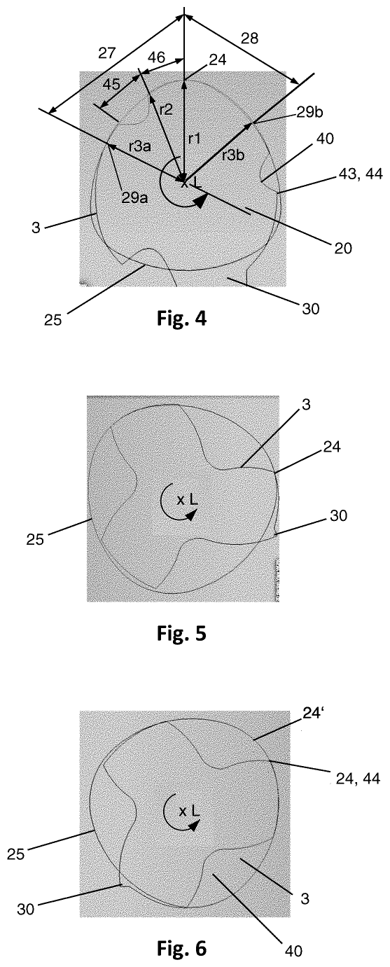

FIGS. 4 to 6 illustrates cross-sectional views of the drill bit 1 at different positions along the longitudinal axis L. The cross-sectional views illustrate cross-sections of the drill bit 1 perpendicular to the longitudinal axis L as viewed in an apical-coronal direction. Each of these cross-sectional views shows an outline 3 of the drill bit 1, wherein the outline may include outline sections originating from features such as a flute 40 or a guiding thread 30.

The cross-sectional views of FIGS. 4 to 6 each include for the sake of comparison a basic outline 25 of the drill bit core 20, i.e., an outline of the drill bit core 20 representing its basic shape without any additional features such as a flute 40 or a guiding thread 30. In the cross-sectional view of FIG. 6, the basic outline 3 allows to indicate, where an outermost point 24′ of the drill bit core 20 without a flute 40 would be located.

An outline 3 of a cross-section of the drill bit core 20 preferably comprises at least one outermost point 24. The at least one outermost point 24 is substantially located at a first radial distance r1 from the longitudinal axis L. Consequently, the radial distance r1 at the outermost point 12 represents a maximum radial distance of a point on the basic outline 25 (or the outline 3) to the longitudinal axis L.

In FIGS. 4 to 6, the basic outline 25 of the cross-sections of the drill bit core 20 comprises three outermost points 24. However, any other number of outermost points 12 may be formed such as one, two (i.e., oval), three (i.e., trioval), four, five, or six outermost points 12. Preferably, the outermost points 12 are uniformly distributed along the outline of the drill bit core 20.

The cross-sectional views of FIG. 4 to 6 each illustrate a cross-section at a different portion of the drill bit 1 along the longitudinal axis L. In these cross-sectional views, a predetermined direction of rotation of the drill bit 1 is defined in a counter-clockwise direction as indicated by the curved arrows.

The drill bit 1 may comprise at least a portion along the longitudinal axis L in an apical-coronal direction, where the diameter and/or the first radial distance r1 of the drill bit core 20 increases. This increase preferably corresponds to an increase in diameter of a dental implant (not shown) to be implanted into the recess created by means of the drill bit 1.

Further, the drill bit 1 may comprise at least a portion along the longitudinal axis L, where the diameter and/or the first radial distance r1 of the drill bit core 20 remains substantially the same. Such a portion may also be shaped to correspond to an implant to be implanted.

At a coronal end 4 of a portion of the drill bit 1 that is configured to interact with bone tissue, the shape of the drill bit 1 may be adapted to have substantially the same size as the dental implant at that location in an implanted state. At the apical end 2, the drill bit 1 may be adapted to be smaller in size than the dental implant. The latter is for enhancing primary stability of the implant by causing a press-fit between the patient's bone tissue and the implant.

Generally, it is advantageous to adapt the shape of the drill bit 1 along the longitudinal axis L to a geometry of a dental implant to be inserted in a bone recess which is to be prepared with the drill bit 1. Preferably, this adaptation takes the type of bone into consideration that will be located adjacent the implant after implantation. In particular in soft bone, the drill bit 1 is undersized in relation to the implant for supporting the anchorage of the implant within bone tissue.

A (mean) size of the drill bit 1 that is basically the same as the dental implant at the coronal end 4 reduces the strain in the cortical bone tissue after implantation to prevent bone resorption and to foster bone ingrowth.

A smaller size of the drill bit 1 in relation to the dental implant at the apical end 2 achieves a good first stability of the dental implant within the bone recess.

A non-circular drill bit core 20 comprises at least one compression zone 27 and at least one relaxation zone 28 (see FIG. 4).

In the predetermined direction of rotation of the drill bit 1, the at least one compression zone 27 extends along a portion of the outline of a cross-section of the drill bit core 20 starting at the innermost point 29a with the radial distance r3a from the longitudinal axis L preferably to an outermost point 24 along the outline and at a maximum radial distance r1 between the outline and the longitudinal axis L.

Also in the predetermined direction of rotation of the drill bit 1, the at least one relaxation zone 28 extends along a portion of the outline of a cross-section of the drill bit core 20 starting at an outermost point 24 at a maximum radial distance r1 from the longitudinal axis L to the innermost point 29b having the radial distance r3b from the longitudinal axis L. The distances r3a and r3b may be equal.

The outline between an innermost point 29 and an outermost point 24 of the compression zone 27 and/or relaxation zone 28 is preferably smooth (e.g. (only) curved). Nonetheless, either or both zones 27 and 28 may have at least one straight subsection.

It should be noted that an outline of the drill bit core's cross-section having more than one outermost point 24 that are located directly adjacent to each other, i.e. they form a circular line section, is also encompassed since all these outermost points 24 have the first radial distance r1 (maximum radial distance). The same applies to the innermost point 29 at the minimum radial distance r3 from the longitudinal axis L. Nonetheless, having singular outermost point(s) 24 and/or innermost point(s) 29 is preferred.

The number of compression zones 27 in the cross-sections of the drill bit core 20 may be generally equal to the number of relaxation zones 28. In this context, the term generally is used since either or both zones 27 and 28 may be interrupted by a cutting zone 45 that will be described in more detail below.

In the compression zone 27, the drill bit 1 is configured to compress bone tissue in a radially outward direction, i.e. to exert a densifying or condensing effect on the bone tissue. Upon rotation of the drill bit, a predetermined point in the bone tissue (not shown) is urged radially outward due to the increasing radial distance of the cross-sections of the drill bit core 20 from the radial distance r3 to the radial distance r1. This outward movement relative to the bone tissue causes the bone tissue to be condensed.

In the relaxation zone, the drill bit 1 allows the compressed bone tissue to recover by the outline 3 of the drill bit core 20 moving radially inward. In other words, upon rotation of the drill bit, a predetermined point in the bone tissue (not shown) is able to follow radially inwards due to the decreasing radial distance of the drill bit core's outline from the outermost point 24 to the innermost point 29.

Without wishing to be bound by theory, it has been found that bone tissue with a high density, e.g. cortical bone, generally recovers faster than bone tissue with a low density, e.g. cancellous bone. In other words, in the relaxation zone of the drill bit 1, bone tissue with a higher density relaxes and extends faster radially inward than bone tissue with a lower density.

At least a part of an outline of the cross-section of the drill bit core 20 is preferably non-circular (but is preferably round or curved). Accordingly, at least a portion of the drill bit core 20 along the longitudinal axis L has an at least partly non-circular cross-section.

An outline of the cross-section of the drill bit core 20 may also be non-circular along its full extension or at least along substantially the full extension of the drill bit core 20 along the longitudinal axis L. For example, the non-circular outline may be oval or trioval. However, other shapes of non-circular outlines are also possible, in particular a substantially round outline. Nonetheless and as previously discussed, the drill bit core 20 may also comprise an outline of the cross-section at least along a portion of the longitudinal axis L, which is substantially circular.

A non-circular outline allows to define at least one compression zone 27 and relaxation zone 28. That is, the portion of the outline of a cross-section of the drill bit core 20 in a predetermined direction of rotation of the drill bit 1 from a minimum radial distance r3a to a maximum radial distance r1 is defined as a compression zone 27, i.e. it is configured to compress adjacent bone tissue. The portion of the outline of a cross-section of the drill bit core 20 in a predetermined direction of rotation of the drill bit 1 from a maximum radial distance r1 to a minimum radial distance r3b is defined as a relaxation zone 28, i.e. it is configured to allow for relaxation of previously compressed bone tissue. The shape of the non-circular outline 3 may be designed so that a plurality of compression zones 27 and relaxation zones 28 along an outline of the cross-section of the drill bit core 20 are defined.

The drill bit 1 may be configured differently at the coronal end 4, where the drill bit 1 is interacting with cortical bone tissue with a compact structure and a comparatively high density, than at the apical end, where the drill bit 1 is generally preparing cancellous bone tissue with a trabecular structure and a generally lower density. Thus, it is possible to prepare these different regions of bone tissue in a single step when preparing the bone recess for inserting a dental implant. In this way, complex drill protocols may be avoided in most patients. Preferably, a pilot drill may be employed prior the drill bit 1 to facilitate preparation.

The drill bit 1 comprises a cutting portion extending at least partially along (preferably along the entire) flute 40. Thus, at least a part of the flute 40 may be configured as a non-cutting flute, i.e. a flute with an edge that does not form a cutting edge 43.

The cutting portion may be present along substantially the full length of the drill bit core 20 along the longitudinal axis L from the apical end 2 to the coronal end 4, i.e., along the portion that is configured to interact with bone tissue.

Preferably, the flute 40 extends, along the longitudinal axis L, over the full length of the (operative part of) drill bit. In other words, a first extremity of at least one flute is at an apical end 2 of the drill bit and a second extremity of said flute is at the coronal end of the drill bit that is configured to interact with bone tissue. The flute 40 can extend continuously between said first and second extremities. The drill bit can comprise two or more flutes having the same above-mentioned features.

As previously described, the cutting portion preferably tapers apically, i.e. the cross-sections of the cutting portion perpendicular to the longitudinal axis L decrease in size from the coronal end of a cutting portion to an apical end of a cutting portion. As a result, when the drill bit drills into the bone tissue, the cutting portion increases the size of the hole that is created by cutting adjacent bone tissue. If a guiding thread 30 is present, the cutting motion is performed with a particular steady feed.

The cutting portion may at least partly or entirely also be configured to condense bone tissue. Accordingly, a cross-section of such a cutting portion comprises a compression zone 27 and a relaxation zone 28. Thereby, such a cutting portion is configured to compress bone tissue in the compression zone 27 and to allow the bone tissue to relax in the relaxation zone 28 while rotating in the predetermined direction of rotation of the drill bit 1.

As mentioned above, the profile along and/or the transition between the compression zone 27 and relaxation zone 28 is preferably (only) curved, i.e. it may not include a straight section or a discontinuity (except for a cutting zone 45). This has a positive effect on the structural integrity of the bone tissue that is treated with the drill bit 1.

Nonetheless, at least a part of or the cutting portion may also be configured as a cutting portion without being configured to condense bone tissue by rotation or in a rotational direction. Such a cutting portion is configured without compression and relaxation zones 27 and 28.

However, the outline of a cross-section of the drill bit core 20 along at least a part of or the entire cutting portion is preferably non-circular and includes a compression zone 27 and a relaxation zone 28. This results in the outline being configured to have a condensing effect on bone tissue upon rotation. Depending on the configuration of the non-circular outline, a plurality of compression zones and relaxation zones along an outline of the cross-section of the drill bit core 20 may be defined.

If not being configured as condensing in a rotational direction, the outline of the cross-section of the drill bit core 20 may be substantially circular. The outline of a cross-section of a cutting portion that is not configured to condense bone tissue in a rotational direction is substantially circular if a flute 40 and/or a guiding thread 30 is present.

Along an outline of a cutting portion's cross-section of the drill bit core 20, the cutting portion preferably comprises at least one cutting zone 45 including a cutting point 44. The cutting zone 45 is defined by a flute 40 forming a concave recess in the outline of the cutting portion and comprising a cutting edge 43. Accordingly, the cutting portion comprises at least one flute 40. As described above, the flute 40 is preferably formed as a recess or groove in and extending along the drill bit core 20. Further, the flute 40 may be straight but preferably extends helically around the drill bit core 20. The latter case distributes the cutting force along the circumference of the drill bit 1 and facilitates guidance during insertion.

Preferably, the cutting zone 45 including the cutting point 44 is located in the compression zone 27 of the outline 3 of a drill bit's cutting portion that is also configured for condensing bone tissue. In this case, the outline of the compression zone 27 is interrupted by the cutting zone 45.

The at least one cutting point 44 is located at a second radial distance r2 from the longitudinal axis L in a cross-sectional view of the drill bit core 20 along the cutting portion. An outline 3 of the cross-sections of the drill bit core 20 along the cutting portion may comprise two or three pairs of outermost points 24 and cutting points 44, wherein each pair comprises an outermost point 24 and a cutting point 44.

A cutting point 44 preferably represents a discontinuity along the outline 3 of a cutting portion. Accordingly, the cutting point 44 comprises a clearance angle, a lip angle, and a rake angle.

The at least one cutting point 44 located at a second radial distance r2 allows to cut bone tissue along a circle within the cross-section having the second radial distance r2 from the longitudinal axis L as a radius. If the second radial distance r2 is smaller than the first radial distance r1 of an outermost point 24 described in more detail below, the drill bit 1 is not configured to cut bone tissue in an area having a radial distance that is larger than the second radial distance r2.

Thus, the second radial distance r2 may be smaller than the first radial distance r1 of the outermost point 24 in a cross-section of the drill bit core 20 in at least a part of the cutting portion along the longitudinal axis L, i.e. at least in some of the cross-sections along the longitudinal axis L. In other words, the drill bit 1 and the cutting portion may comprise a portion along the longitudinal axis L with a negative clearance angle.

Alternatively, the first radial distance r1 and the second radial distance r2 may also be substantially equal in a cross-section of the drill bit core 20 in at least a portion of the cutting portion along the longitudinal axis L. In other words, the outermost point 24 and the cutting point 44 may substantially coincide. In this case, the drill bit 20 and the cutting portion may comprise a portion along the longitudinal axis, where the cutting point 44 has a positive clearance angle.

The ratio between the first radial distance r1 of the outermost point 24 and the second radial distance r2 of the cutting point 44 may change from cross-section to cross-section of the drill bit core 20 along the cutting portion.

In an embodiment, where the second radial distance r2 is smaller than the first radial distance r1, the drill bit 1 and the cutting portion comprise a non-cutting zone 46 where the drill bit 1 is not configured to cut bone (cf. cross-section of FIG. 4).

The radial extension of the non-cutting zone 46 defines a ring-shaped area between the second radial distance r2 and the first radial distance r1. Within this non-cutting zone 46, the drill bit 1 is configured to alternately compress or allow for relaxation of bone tissue, i.e. to exert a force on the bone tissue in the radial direction without cutting the bone tissue. As described above, in the compression zone 27, bone tissue adjacent to the compression zone is urged radially outward upon rotation of the drill bit 1 in the predetermined direction of rotation, whereas in the relaxation zone 28, a recovery of bone tissue in a direction radially inward is allowed.

Without wishing to be bound by theory, the drill bit 1 takes advantage of the observation that bone tissue with a greater density recovers faster, i.e. moves faster radially inward, than bone tissue with a lower density. Subsequently to compressing bone tissue in the compression zone 27 in a direction radially outward, the compressed bone tissue recovers radially inward in the relaxation zone 28. Due to the difference in recovery time, the drill bit 1 has a bias toward cutting bone tissue with a greater density (e.g. cortical bone tissue).

The time allowed for recovery is dependent on the circumferential position of the cutting point 44 and the outermost point 24 (and the rotation rate and/or speed of the drill bit 1) and, thus, the location of the compression zone 27 and the relaxation zone 28 along the outline 3 of a cross-section of the drill bit core 20 in relation to the cutting point 44. Bone tissue, which recovers in the predetermined allowed recovery time to a radial distance smaller than the second radial distance r2 is to be cut at the next cutting point 44 passing by, whereas bone tissue which recovers to a point between the second radial distance r2 and the first radial distance r1 is not to be cut at the next cutting point 44. Thus, the cutting point 44 is able to cut bone tissue of a varying degree according to its properties, i.e. to cut hard bone tissue to a higher degree than soft bone tissue. This effect can be adjusted via the geometry of the drill bit core 20, i.e., the magnitude of the first and second radial distances r1 and r2 as well as the circumferential positions thereof.

In a case, in which the second radial distance r2 is substantially equal to the first radial distance r1, the drill bit 1 and the cutting portion have a cutting behavior that is different from the previously described cutting behavior. Since the cutting point 44 and the outermost point 24 substantially coincide, the cutting point 44 is located at the most radial outward position. In other words, all other points on an outline of the cross-section of the drill bit core 20 are located more radially inward than the cutting point 44 (see FIGS. 5 and 6). Therefore, the drill bit 1 is not configured to compress or allow for relaxation of bone tissue but, instead, cuts bone tissue in a circular area defined by the first radial distance r1 or the second radial distance r2.

This is structurally illustrated in FIGS. 5 and 6. In the cross-section of FIG. 6, the theoretical outermost point 24′ of the basic outline 25 of the drill bit core 20 (i.e., the outline not considering a guiding thread 30 and/or a cutting flute 40) as well as the outermost point 24 of the drill bit's outline 3 coincide with the cutting point 44 of the drill bit's outline 3.

It should be noted that the determination of the outermost point 24 of the drill bit's outline 3 does not take a guiding thread 30 into account, if present (cf. cross-sections of FIGS. 4 to 6). Further, albeit cross-sections perpendicular to the longitudinal axis along the cutting portion may generally comprise a predetermined number of compression zones 27, relaxation zones 28, cutting zones 45, non-cutting zones 46, outermost points 24, and/or cutting points 44, certain cross-sections may not include all of these features due to an influence of other structural features of the drill bit 10, in particular a guiding thread 30 (cf. FIG. 4 missing one relaxation zone and part of one compression zone due to the guiding thread 30).

Turning to FIG. 6, the theoretical outermost point 24′ of the basic outline 25 of the drill bit core 20 is located along the cutting zone 45, where the flute 40 along the outline 3 of the drill bit 1 is formed. Thus, the theoretical outermost point 24′ does not coincide with the outermost point 44 of the drill bit's outline 3. However, also in this case the outermost point 24 coincides with the cutting point 44.

It should be noted that the cross-section of FIG. 6 does not include a compression zone 27 or a relaxation zone 28. Although a part of the outline corresponds structurally to a relaxation zone 28, it fails to act as one in terms of its functional configuration. In other words, the outline 3 lacks a compression zone 27 having the function to compress bone tissue so that there cannot be a relaxation zone 28 that allows for a relaxation of bone tissue that has previously been compressed by a compression zone 27 of the drill bit 1.

The skilled person will appreciate from the explanation above that the cutting behavior of the drill bit 1 changes with a change in the ratio between the first radial distance r1 and the second radial distance r2. Accordingly, it is possible to adjust the cutting behavior of the drill bit 1 along the longitudinal axis L, in particular taking different regions in the depth direction of bone tissue at an implantation site into account.

Preferably, a cutting portion comprises a first part along the longitudinal axis L with the second radial distance r2 of the cutting point 44 being smaller than the first radial distance r1 of the outermost point 24. In this first part, the cutting portion may comprise a negative clearance angle at the cutting point 14. Further, the first part of the cutting portion may also be configured to condense bone tissue upon rotation. Accordingly, an outline of a cross-section of the first part of the cutting portion preferably comprises at least one compression zone 27 and at least one relaxation zone 28.

The cutting portion may further comprise a second part. In the second part of the cutting portion, a second radial distance r2 may substantially be equal to the first radial distance r1. The second part of the cutting portion may further comprise a cutting point 44 with a positive clearance angle. Preferably, the first part of the cutting portion is positioned apically to the second part of the cutting portion.

Due to the difference in ratio of the first radial distance r1 and the second radial distance r2 between the first part and the second part of the cutting portion, the cutting behavior of these parts differ from each other.

In particular, the outline of the first part of the cutting portion is configured, upon rotation of the drill bit, to compress bone tissue in the compression zone 27, allow for relaxation of the bone tissue in the relaxation zone 28, and then cut the bone tissue at the cutting point 44 of the cutting zone 45. As a result, the amount of bone tissue being cut upon rotation depends on the relaxation properties of the bone tissue, i.e. how much and how fast does the bone tissue relax after being compressed.

Without wishing to be bound by theory, the inventors observed that cancellous bone with a relatively low bone density is cut less. Accordingly, more of the soft bone tissue remains for supporting the dental implant to be inserted.

The second part of the cutting portion is not configured to compress bone tissue upon rotation but, instead, cuts bone tissue at the outermost point 24 corresponding to the cutting point 44.

Without wishing to be bound by theory, this feature is advantageous in the cortical bone with a relatively high bone density where further condensing of bone tissue has less effect. Accordingly, the second part preferably creates a bone recess in the cortical region of the bone tissue with a size substantially corresponding to the size of a dental implant to be implanted for avoiding bone resorption and, thus, a faster ingrowth of the dental implant.

In the predetermined direction of rotation of the drill bit 1, the cutting point 44 along the first part may be located along the outline of the drill bit's cross-section so that upon rotation the cutting point 44 passes a bone tissue location before or at the same time as the outermost point 24. After the relative position of the outermost point 24 and the cutting point 44 has changed along the outline so that they correspond to each other, the theoretical outermost point 24′ may continue to change its relative position to pass a bone tissue location before the cutting point. This causes a continuous change in the clearance angle from a negative to a positive clearance angle.

As explained above, the geometry of the drill bit 1 and, in particular, the magnitude of the first and second radial distances r1 and r2 as well as the circumferential positions thereof define the cutting behavior of the drill bit 1.

As mentioned above, the cutting flute may extend helically around the drill bit 1. Accordingly, the cutting points 44 also extend helically around the drill bit 1, in particular with a first pitch. The first pitch may be the same pitch as the pitch of the cutting flute 15 (cutting flute with a constant size) or may be different than the pitch of the cutting flute (cutting flute with a changing size). In particular, the first pitch may be smaller than the pitch of the cutting flute (i.e. the size of the cutting flute increases in an apical-coronal direction).

For a change of the cutting behavior, the outermost points 24 of the cross-sections of the drill bit core 20 along the longitudinal axis L may be located along a helical line with a second pitch around the longitudinal axis L. Alternatively, the outermost points 24 of these cross-sections may also be located along a substantially straight line that is preferably arranged parallel to the longitudinal axis L.

Preferably, the first pitch and the second pitch of the cutting points 44 and the outermost points 24, respectively differ from each other. In particular, the first pitch is smaller than the second pitch. Alternatively, the first and second pitch may substantially be equal.

In an embodiment where the first pitch and the second pitch differ from each other, the relative circumferential position of the outermost point 24 and the cutting point 44 changes along the longitudinal axis L of the cutting portion of the drill bit 1. It is thus possible to continuously adjust the cutting behavior of the drill bit 1 along the longitudinal axis L to different regions of the bone.

With the drill bit 1 according to the present disclosure, it is possible to reduce the complexity of a drill protocol. A sequential use of a plurality of different tools and complex drill protocols may thus be avoided and a misalignment of sequentially used tools may be more easily prevented. Further, the drill bit 1 allows to prevent the bone recess to be formed from being oversized or undersized.

### REFERENCE SIGNS

The following lists reference signs used in the description and the drawings. Throughout the drawings these reference signs refer to features that have the same or an equivalent function and/or structure.
1 drill bit
2 apical end
3 outline
4 coronal end of bone interacting portion
10 apical end face
11 central area
12 arm-shaped radial extension of apical end face
14 circumferential edge of apical end face
14a tip of an arm of apical end face
14b side edge of an arm of apical end face
14c opposite side edge of an arm of apical end face
20 drill bit core
21 circumferential surface of the drill bit core
22 apical edge of drill bit core
23 chamfer
24 outermost point
24′ theoretical outermost point
25 basic outline of the drill bit core
27 compression zone
28 relaxation zone
29 innermost point
30 guiding thread
40 flute
41 concave surface
42 apical edge of concave surface
43 cutting edge
44 cutting point
45 cutting zone
46 non-cutting zone
50 retention arm
r1 first radial distance
r2 second radial distance
r3 smallest radial distance
L longitudinal axis

1. A drill bit (1), comprising:
an apical end (2), a coronal end, and a longitudinal axis (L) extending between the apical end and the coronal end,
a drill bit core (20) extending along the longitudinal axis (L),
at least two flutes (40), each flute being defined by a concave surface (41) forming a recess along the drill bit core (20); and
an apical end face (10) at the apical end (2), wherein
apical edges (42) of the concave surfaces (41) and
an apical edge (22) of the drill bit core (20)
define the shape of the apical end face so that the apical end face defines a profile of retention arms (50) at the apical end (2), each retention arm being formed between the concave surfaces of adjacent flutes (40)
and wherein
at least one of the at least two flutes (40) extends along the longitudinal axis (L),
over the full length of the drill bit (1) configured to interact with bone tissue.

2. The drill bit (1) of claim 1, further comprising:
a cutting portion extending along the entirety of at least one of the at least two flutes (40), said cutting portion being present along substantially the full length of the drill bit core (20) along the longitudinal axis (L) from the apical end (2) to the coronal end of the portion of the drill bit configured to interact with bone tissue.

3. The drill bit (1) according to claim 1, further comprising at least one guiding thread (30) protruding from the drill bit core (20) and helically extending along the drill bit core (20), the guiding thread (30) tapering apically.

4. The drill bit (1) according to claim 3, wherein the radial height of the guiding thread (30) is smaller than the radial extension of the retention arm's profile at the apical end face (10).

5. The drill bit (1) according to claim 3, wherein the guiding thread (30) has a non-circular outer circumferential profile, the non-circular outer circumferential profile being a trioval profile.

6. The drill bit (1) according to claim 1, wherein each retention arm (50) has a radial extension that is greater than its circumferential width.

7. The drill bit (1) according to claim 1, wherein a circumferential edge (14) of the apical end face (10) is arranged in a plane perpendicular to the longitudinal axis (L).

8. The drill bit (1) according to claim 1, wherein the drill bit core (20) has a non-circular profile in a cross-section perpendicular to the longitudinal axis (L), wherein in a longitudinal direction of the drill bit (1), the non-circular profile rotates around the longitudinal axis (L) creating a twisted non-circular drill bit core.

9. The drill bit (1) according to claim 1, wherein the drill bit core (20) is tapered towards the apical end (2).

10. The drill bit (1) according to claim 1, wherein the concave surface (41) of each flute (40) and the circumferential surface (21) of the drill bit core (20) form a cutting edge (43).

11. The drill bit (1) according to claim 1, wherein at the apical edge of each concave surface (41), the concave surface and the apical end face (10) partially form a cutting edge, the cutting edge having a forward rake angle and substantially no forward relief angle.

12. The drill bit (1) according to claim 1, wherein the at least two flutes (40) extend helically along the drill bit core (20).

13. The drill bit (1) according to claim 16, wherein the non-circular profile of the drill bit core (20) and the non-circular outer circumferential profile of the guiding thread (30) are formed with a circumferential offset.

14. A method to create an osteotomy using a drill bit (1), wherein the method comprises:
drilling a hole with a pilot drill;
increasing the size of the hole for the insertion of a dental implant using the drill bit (1) according to claim 1,
wherein upon contact of the apical end face (10) with the bottom of the hole drilled with the pilot drill, the retention arms (50) generate a braking force for stopping the rotation of the drill bit (1).

15. The method according to claim 14, wherein a tip of the pilot drill has a diameter greater than the diameter of the apical end face (10).

16. The drill bit (1) according to claim 1, further comprising at least one guiding thread (30) protruding from the drill bit core (20) and helically extending along the drill bit core (20), the guiding thread (30) tapering apically,
wherein the guiding thread (30) has a non-circular outer circumferential profile, the non-circular outer circumferential profile being a trioval profile, and
wherein the drill bit core (20) has a non-circular profile in a cross-section perpendicular to the longitudinal axis (L), wherein in a longitudinal direction of the drill bit (1), the non-circular profile rotates around the longitudinal axis (L) creating a twisted non-circular drill bit core.

17. A drill bit (1), comprising:
an apical end (2), a coronal end, and a longitudinal axis (L) extending between the apical end and the coronal end,
a drill bit core (20) extending along the longitudinal axis (L),
at least two flutes (40), each flute being defined by a concave surface (41) forming a recess along the drill bit core (20); and
an apical end face (10) at the apical end (2), wherein
apical edges (42) of the concave surfaces (41) and
an apical edge (22) of the drill bit core (20)
define the shape of the apical end face so that the apical end face defines a profile of retention arms (50) at the apical end (2), each retention arm being formed between the concave surfaces of adjacent flutes (40),
the drill bit further comprising a cutting portion extending along the entirety of at least one of the at least two flutes (40), said cutting portion being present along substantially the full length of the drill bit core (20) along the longitudinal axis (L) from the apical end (2) to the coronal end of the portion of the drill bit configured to interact with bone tissue.

18. The drill bit (1) according to claim 17, further comprising at least one guiding thread (30) protruding from the drill bit core (20) and helically extending along the drill bit core (20), the guiding thread (30) tapering apically.

19. The drill bit (1) according to claim 17, wherein each retention arm (50) has a radial extension that is greater than its circumferential width.

20. The drill bit (1) according to claim 17, wherein the drill bit core (20) has a non-circular profile in a cross-section perpendicular to the longitudinal axis (L), wherein in a longitudinal direction of the drill bit (1), the non-circular profile rotates around the longitudinal axis (L) creating a twisted non-circular drill bit core.
